SEQUENCE LISTING (4 sequences)

SEQ ID NO: 1 - DNA, 61 bp, Artificial
  MiRNA sequence for GPD1- knockdown
gaaagttgggtgtcctgcatcaggttttggccactgactgacctgatgcacacccaacttt

SEQ ID NO: 2 - DNA, 20 bp, Artificial
  CRISPR/Cas oligonucleotide sgRNA1
aaagtctgcattgtcggctc

SEQ ID NO: 3 - DNA, 20 bp, Artificial
  CRISPR/Cas oligonucleotide sgRNA2
tgcattgctacccacgatct

SEQ ID NO: 4 - DNA, 20 bp, Artificial
  CRISPR/Cas oligonucleotide, human sequence
gagaatgtcaaatacctgcc

(19) United States
(12) Patent Application Publication
(10) Pub. No.: US 2019/0276833 A1
(43) Pub. Date: Sep. 12, 2019

(54) CANCER TREATMENT BY GLYCEROL-3-PHOSPHATE DEHYDROGENASE 1 INHIBITION

(71) Applicant: Deutsches Krebsforschungszentrum, Heidelberg (DE)

(72) Inventors: Patricia Rusu, Vermont (AU); Liu Hai-Kun, Weinheim (DE)

(21) Appl. No.: 16/462,052

(22) PCT Filed: Nov. 14, 2017
(86) PCT No.: PCT/EP2017/079158

§ 371 Date: May 17, 2019

(30) Foreign Application Priority Data
Nov. 17, 2016 (EP) 16199331.6

(51) Int. Cl. C12N 15/113; A61K 45/06; C12Q 1/6886; C12Q 1/32; C07K 16/40; A61K 31/353; C12Q 1/66

(52) U.S. Cl. CPC C12N 15/1137; G01N 2333/902; C12Q 1/6886; C12Q 1/32; C07K 16/40; A61K 31/353; C12Q 1/66; C12N 2310/141; C12N 2310/11; C12N 2310/122; C12N 2310/14; C12N 2310/12; C12N 2310/20; C12Q 2600/106; C12Q 2600/158; A61K 45/06

(57) ABSTRACT

The present invention relates to an inhibitor of glycerol-3-phosphate dehydrogenase 1 (GPD1 inhibitor) for use in treatment and/or prevention of cancer. The present invention further relates to a kit comprising a GPD1 inhibitor comprised in a housing; to a method for identifying a subject suffering from cancer susceptible to cancer treatment by administration of a GPD1 inhibitor comprising a) determining in a sample of said subject the amount of a GPD1 gene product, b) comparing said amount determined in step a) to a reference, and c) based on the result of step b), identifying a subject susceptible to cancer treatment by administration of a GPD1 inhibitor; and to further means and methods related thereto.

The present invention relates to an inhibitor of glycerol-3-phosphate dehydrogenase 1 (GPD1 inhibitor) for use in treatment and/or prevention of cancer. The present invention further relates to a kit comprising a GPD1 inhibitor comprised in a housing, to a method for identifying a subject suffering from cancer susceptible to cancer treatment by administration of a GPD1 inhibitor comprising a) determining in a sample of said subject the amount of a GPD1 gene product, b) comparing said amount determined in step a) to a reference, and c) based on the result of step b), identifying a subject susceptible to cancer treatment by administration of a GPD1 inhibitor; and to further means and methods related thereto.

Cancer constitutes the fourth leading cause of death in Western countries. As the average age in the Western population steadily rises, so do cancer-related deaths indicating that cancer will be one of the most common causes of death in the 21st century. The aggressive cancer cell phenotype is the result of a variety of genetic and epigenetic alterations leading to deregulation of intracellular signaling pathways. Cancer cells commonly fail to undergo so-called “programmed cell death” or “apoptosis”, a signaling process that plays a key role in preventing cell tissues from abnormal growth.

Three modes of cancer therapy are generally available. Curative surgery attempts to remove the tumor completely. This is only possible as long as there are no metastases. Sometimes surgery may be an option for the treatment of metastases if there are only few and they are easily accessible. Radiotherapy uses ionizing radiation, typically γ-radiation, to destroy the tumor. Radiation therapy is based on the principle that tumor cells with their high metabolic rates are especially susceptible to radiation induced cell damage. The anti-tumor effect of radiation therapy has to be weighed against the damage to the surrounding healthy tissue. Thus, possible tissue damage can rule out this option in some cases due to the damage to healthy tissues to be feared. Furthermore, radiation therapy is limited to cases where the primary tumor has not yet spread or where only few metastases are present.

The most commonly used—and in many instances the only available—systemic treatment for cancer is chemotherapy. For patients suffering from leukemia or from metastases of solid tumors, thus, chemotherapy is the only treatment option. Chemotherapeutic agents are cytotoxic for all rapidly dividing cells. As cancer cells usually divide more rapidly than other cells in the body, they are preferably killed by those agents. Common groups of chemotherapeutic agents are substances that inhibit cell division by interfering with the formation of the mitotic spindle or agents, which damage the DNA, e.g. by alkylating the bases. As chemotherapeutic agents target all rapidly dividing cells, their side effects are usually severe. Depending on the substance used, they include organ toxicity (e.g. heart or kidney), immunosuppression, neurotoxicity and anemia. Some groups of chemotherapeutic agents, e.g. alkylating agents, even have the potential to cause cancer. Due to these side effects, dosages have sometimes to be reduced or chemotherapy has to be discontinued completely. Furthermore, the side effects of chemotherapy often prohibit the treatment of patients in a bad general condition. Adding to all these problems is the often limited efficacy of chemotherapy. In some cases chemotherapy fails from the very beginning. In other cases, tumor cells become resistant during the course of treatment. To combat the emergence of resistant tumor cells and to limit the side effects of chemotherapy, combinations of different compounds with different modes of action are used. Nevertheless, the success of chemotherapy has been limited, especially in the treatment of solid tumors.

Recently, drugs have become available whose mode of action is not based on toxicity against rapidly dividing cells. These compounds show a higher specificity for cancer cells and thus fewer side effects than conventional chemotherapeutic agents. Imatinib, for example, is used for the specific treatment of chronic myelogenous leukemia. This compound specifically inhibits an abnormal tyrosine kinase, which is the product of a fusion gene of bcr and abl. Because this kinase does not occur in non-malignant cells, treatment with Imatinib has only mild side effects. However, Imatinib is not used for the treatment of hematological cancers other than myelogenous leukemia. Rituximab is a monoclonal antibody directed against the cluster of differentiation 20 (CD20), which is widely expressed on B-cells. It is used for the treatment of B cell lymphomas in combination with conventional chemotherapy.

Primary brain tumors can be benign or malignant. Of malignant primary brain tumors, glioblastoma, in particular glioblastoma multiforme (GBM) is the most common and the most aggressive form. GBM is known to contain stem cell like cells having increased propensity for self-renewal (Murat et al. (2008), Journal of Clinical Oncology 26 (18): 3015), which is why these cells are viewed as a cause for frequent relapse in this type of cancer. Over time, stem cell like cells have been identified also in other tumors, including brain, breast, colon, ovary, pancreas, prostate, melanoma, multiple myeloma, and non-melanoma skin cancer.

Glycerol-3-phosphate dehydrogenase (GPD1) is a cytosolic enzyme that catalyzes the reversible redox reaction of dihydroxyacetonephosphate and NADH to glycerol-3-phosphate (G3P) and NAD+. GPD1 is required to prevent the accumulation of dihydroxyacetonephosphate and to replenish the NAD+ pool in the cytosol. Thus, GPD1 is an important link between carbohydrate and lipid metabolism, as glycerol produced from glycerol-3-phosphate is a building block for lipids. Together, GPD1 in the cytosol and GPD2 (also known as GPDH-M) in the mitochondria, constitute the G3P shuttle, which, as a net reaction, transports redox equivalents into mitochondria, where they are oxidized (Stryer et al. (2002), Biochemistry). Interestingly, the cytosolic and mitochondrial iso forms of GPD (GPD1 vs. GPD2) are not structurally related.

GPD1 homozygous mutant mice have no phenotype (Prochazka et al. (1989), The Journal of Biological Chemistry March 15; 264(8):4679-83). GPD1 is highly expressed in liver, muscle, adipose tissue and intestine, but has a low expression in other tissues (Gao et al. (2011), Molecular Biology Reports March; 38(3):1875-81). Overexpression of GPD1 is associated with obesity and increased lipid storage (Swierczynski et al. (2003), Molecular and Cellular Biochemistry December; 254(1-2):55-9.).

In view of the above, there is a need in the art for providing improved means and methods for treating and/or preventing cancer, avoiding the drawbacks of the prior art, and in particular to inhibit or remove cancer stem cells from a patient. It is therefore an objective of the present invention to provide means and methods related to treatment of cancer which fully or partially avoids the short-comings of the prior art.

Accordingly, the present invention relates to an inhibitor of glycerol-3-phosphate dehydrogenase 1 (GPD1 inhibitor) for use in treatment and/or prevention of cancer.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding further embodiments of the invention, without any restrictions regarding the scope of the invention and without any restriction regarding the possibility of combining the features introduced in such way with other optional or non-optional features of the invention. Moreover, if not otherwise indicated, the term “about” relates to the indicated value with the commonly accepted technical precision in the relevant field, preferably relates to the indicated value ±20%, more preferably ±10%, most preferably ±5%.

The terms “glycerol-3-phosphate dehydrogenase 1” and “GPD1” relate to a cytosolic enzyme of multicellular organisms catalyzing the reversible redox reaction of dihydroxyacetonephosphate (glyceronephosphate) and NADH to glycerol-3-phosphate (G3P) and NAD⁺ (EC 1.1.1.8). Preferably, the GPD1 is an animal GPD1, more preferably a mammalian GPD1, even more preferably a human GPD1. Most preferably, the GPD1 is the human GPD1 comprising the amino acid sequence of Genbank Acc No: NP_005267.2 GI:33695088 (isoform 1), preferably encoded by a polynucleotide comprising the nucleic acid sequence of Genbank Acc No: NM 005276.3 GI:380714666 and/or the amino acid sequence of Genbank Acc No: NP 001244128.1 GI:380714668 (isoform 2), preferably encoded by a polynucleotide comprising the nucleic acid sequence of Genbank Acc No: NM_001257199.1 GI:380714667. Preferably, said human GPD1 is encoded by a polynucleotide comprising the nucleic acid sequence of Genbank Acc No: NG_032168.1 GI:381140032.

The terms “inhibitor of glycerol-3-phosphate dehydrogenase 1” and “GPD1 inhibitor” relate to a compound significantly reducing, preferably abolishing, activity of glycerol-3-phosphate dehydrogenase 1 in a target cell, wherein “significantly reducing activity” preferably relates to a reduction of measurable activity by at least 50%, more preferably by at least 75%, most preferably by at least 90%. Preferably, the GPD1 inhibitor is a specific GPD inhibitor, i.e. is an inhibitor inhibiting only glycerol-3-phosphate dehydrogenase activity. More preferably, the GPD1 inhibitor is a specific GPD1 inhibitor, i.e. is an inhibitor inhibiting only GPD1 activity as specified elsewhere herein; even more preferably, the specific GPD1 inhibitor inhibits GPD1-like protein activity only by at most 50%, preferably at most 25%, more preferably by at most 10% at a concentration inhibiting GPD1 by 90%. The GPD1-like protein is known to the skilled person; the human GPD1-like protein sequence is available under Genbank Acc. No: Q8N335.1 GI:74750945. The activity of an inhibitor of GPD1 is, preferably, determined in vitro by assaying the enzymatic activity of GPD1 as specified elsewhere herein. Also preferably, the activity of an inhibitor of GPD1 is determined in vivo or in cultured cells by determining the intracellular glycerol-3-phosphate concentration in a cell contacted to a putative or known inhibitor of GPD1. As shown elsewhere herein, if a tumor cell is contacted with an inhibitor of GPD1, the intracellular concentration of glycerol-3-phosphate decreases to a value corresponding to a value measurable in a normal cell. Also preferably, the activity of an inhibitor of GPD1 is determined in vivo or in cultured cells by determining intracellular lipid droplet accumulation, preferably as shown in the Examples, e.g. by staining with a lipophilic dye, preferably with oil red 0. As shown elsewhere herein, if a tumor cell is contacted with an inhibitor of GPD1, the intracellular concentration of lipids, in particular triacylglycerols, increases compared to a value corresponding to a value measurable in a normal cell.

Preferably, the inhibitor of glycerol-3-phosphate dehydrogenase 1 is a direct inhibitor of glycerol-3-phosphate dehydrogenase 1, i.e., preferably, is a compound directly interacting with GPD1 and, thereby, inhibiting GPD1 activity. Preferably, the direct inhibitor of GPD1 is a polypeptide or polynucleotide specifically binding GPD1 and inhibiting GPD1. More preferably, the direct inhibitor of GPD1 is a polypeptide specifically binding and inhibiting GPD1, i.e. is an inhibitory polypeptide. More preferably, the direct inhibitor of GPD1 is a polypeptide specifically binding and inhibiting GPD1 selected from the list consisting of an antibody, a peptide aptamer, an anticalin, and a Designed Ankyrin Repeat Protein (DARPin), most preferably is an antibody. Also more preferably, the direct inhibitor of GPD1 is a polynucleotide specifically binding and inhibiting GPD1, preferably a polynucleotide aptamer. Preferably, the direct inhibitor of GPD1 is a compound binding to at least one epitope in GPD1, preferably an epitope including at least one amino acid of the active center of GPD1.

The skilled person is aware of methods suitable for determining binding of a direct inhibitor to GPD1, e.g. staining of GPD1-positive cells or of extracts from such cells with a direct inhibitor, wherein said inhibitor is coupled to a detectable label, preferably a colored and/or fluorescent dye; ELISA methods; surface plasmon resonance methods, and the like.

As used herein, the term “antibody” relates to a soluble immunoglobulin from any of the classes IgA, IgD, IgE, IgG, or IgM, having the activity of directly interacting with GPD1 and inhibiting GPD1 activity as specified herein above. Antibodies against GPD1 can be prepared by well-known methods using a purified GPD1 polypeptide or a suitable fragment derived therefrom as an antigen. A fragment, which is suitable as an antigen may be identified by antigenicity determining algorithms well known in the art. Such fragments may be obtained either from the GPD1 polypeptide by proteolytic digestion, may be a synthetic peptide, or may be recombinantly expressed. Suitability of an antibody thus generated as an inhibitor of GPD1 can be tested by an assay as described elsewhere herein. Preferably, the antibody of the present invention is a monoclonal antibody, a human, or humanized antibody, or primatized, chimerized or fragment thereof. More preferably, the antibody is a single chain antibody, a single-domain antibody, a nanobody, or an antibody fragment, such as Fab, scFab, and the like. Also comprised as antibodies of the present invention are a bispecific antibody, a synthetic antibody, or a chemically modified derivative of any of the aforesaid antibodies. Preferably, the antibody of the present invention shall specifically bind (i.e. does not cross react with other polypeptides or peptides) to a GPD1 polypeptide as specified above. Specific binding can be tested by various well-known techniques. Antibodies or fragments thereof can be obtained by using methods, which are described, e.g., in Harlow and Lane “Antibodies, A Laboratory Manual”, CSH Press, Cold Spring Harbor, 1988. Monoclonal antibodies can e.g. be prepared by the techniques originally described in Köhler and Milstein, Nature. 1975. 256: 495; and Galfré, Meth. Enzymol. 1981, 73: 3, which comprise the fusion of mouse myeloma cells to spleen cells derived from immunized mammals. Preferably, the antibody is an antibody as specified above or a polypeptide derivative thereof; more preferably, the antibody is an antibody as specified above.

In the context of this invention, an “aptamer” is a polynucleotide or polypeptide binding specifically to a target molecule by virtue of its three-dimensional structure. Preferably, the aptamer of the present invention is a peptide aptamer. According to the present invention, a “peptide aptamer” is a peptide specifically interacting with GPD1 and, thereby, inhibiting GPD1 activity as specified herein above. Peptide aptamers, preferably, are peptides comprising 8-80 amino acids, more preferably 10-50 amino acids, and most preferably 15-30 amino acids. They can e.g. be isolated from randomized peptide expression libraries in a suitable host system like baker's yeast (see, for example, Klevenz et al., Cell Mol Life Sci. 2002, 59: 1993-1998). A peptide aptamer, preferably, is a free peptide; it is, however, also contemplated by the present invention that a peptide aptamer is fused to a polypeptide serving as “scaffold”, meaning that the covalent linking to said polypeptide serves to fix the three-dimensional structure of said peptide aptamer to a specific conformation. More preferably, the peptide aptamer is fused to a transport signal, in particular a cell-penetrating peptide. Preferably, the aptamer is an aptamer as specified above or a polypeptide or polynucleotide derivative thereof; more preferably, the aptamer is an aptamer as specified above.

As used herein, the term “anticalin” relates to an artificial polypeptide derived from a lipocalin specifically binding GPD1 and inhibiting GPD1 activity. Similarly, a “Designed Ankyrin Repeat Protein” or “DARPin”, as used herein, is an artificial polypeptide, comprising several ankyrin repeat motifs, specifically binding GPD1 and inhibiting GPD1 activity. Preferably, the anticalin or DARPin is an anticalin or DARPin as specified above or a polypeptide derivative thereof; more preferably, the anticalin or DARPin is an anticalin or DARPin as specified above.

Preferably, the inhibitory polypeptide comprises further amino acids which may serve e.g. as immunogens, as a tag for purification and/or detection, or as a linker. In a preferred embodiment of the inhibitory polypeptide of the present invention, said inhibitory polypeptide further comprises a detectable tag. The term “detectable tag” refers to a stretch of amino acids, which are added to or introduced into the inhibitory polypeptide of the invention. Preferably, the tag shall be added C- or N-terminally to the inhibitory polypeptide of the present invention. The said stretch of amino acids shall allow for detection of the inhibitory polypeptide by an antibody which specifically recognizes the tag or it shall allow for forming a functional conformation, such as a chelator or it shall allow for visualization, e.g. in the case of fluorescent tags. Preferred tags are the Myc-tag, FLAG-tag, 6-His-tag, HA-tag, GST-tag or GFP-tag. These tags are all well known in the art. More preferably, the inhibitory polypeptide comprises further amino acids or other modifications, which may serve as mediators of secretion or as mediators of blood-brain-barrier passage.

Preferably, the inhibitor of GPD1 as described herein above is a polypeptide expressible from at least one transcription unit. Thus, preferably, the inhibitor of GPD1 is an antibody, more preferably a monoclonal antibody. More preferably, the inhibitor of GPD1 is a polypeptide expressible from a single transcription unit. Accordingly, preferably, the inhibitor of GPD1 is a polypeptide or a fusion polypeptide. More preferably, the inhibitor of GPD1 is a single chain antibody, a single chain Fab polypeptide, or a nanobody.

Green tea catechins are known to be inhibitors of GPD1 activity (Kao et al. (2010), Planta Med. 76(7):694-6). Thus, preferably, the direct inhibitor of GPD1 is (−)-epicatechin, (−)-epicatechin-3-gallate, (−)-epigallocatechin, (−)-epigallocatechin-3-gallate, more preferably it is (−)-epigallocatechin-3-gallate.

Also preferably, the GPD1 inhibitor is an indirect GPD1 inhibitor, i.e. a compound not directly inhibiting GPD1 activity, but still significantly reducing, preferably preventing, GPD1 activity in a target cell. Preferably, the indirect GPD1 inhibitor specifically binds to a GPD1 encoding polynucleotide, preferably thereby significantly reducing, more preferably preventing, GPD1 expression.

Preferably, the indirect GPD1 inhibitor is a polynucleotide, preferably a polynucleotide inhibiting expression or inducing degradation of a GPD1 mRNA. More preferably, the indirect GPD1 inhibitor is selected from the group consisting of an shRNA, an siRNA, a ribozyme, an antisense molecule, an inhibitory oligonucleotide, and a micro RNA. Most preferably, the indirect GPD1 inhibitor is an shRNA.

It is understood by the skilled person that inhibition of expression or induction of degradation of a specific RNA can be achieved in various ways. It is also understood by the skilled person that the exact embodiment of a polynucleotide being an indirect GPD1 inhibitor of the present invention will depend on the method intended.

Preferably, the indirect GPD1 inhibitor is a ribozyme. The term “ribozyme” as used herein refers to catalytic RNA molecules possessing a well defined tertiary structure that allows for catalyzing either the hydrolysis of one of their own phosphodiester bonds (self-cleaving ribozymes), or the hydrolysis of bonds in other RNAs, but they have also been found to catalyze the aminotransferase activity of the ribosome. The ribozymes envisaged in accordance with the present invention are, preferably, those, which specifically hydrolyse the target RNAs, preferably GPD1 RNA, i.e, preferably RNA transcribed from a GPD1 gene as specified above. In particular, hammerhead ribozymes are preferred in accordance with the present invention. How to generate and use such ribozymes is well known in the art (see, e.g., Hean & Weinberg (2008), RNA and the Regulation of Gene Expression: A Hidden Layer of Complexity, Chapter 1. Caister Academic Press).

Also preferably, the indirect GPD1 inhibitor is a polypeptide comprising a lysosome-degradation sequence, preferably a chaperone-mediated autophagy-targeting motif (CTM). Preferably, said CTM-comprising polypeptide specifically binds to GPD1; e.g. the CTM-comprising polypeptide may further comprise an antibody specifically binding to GPD1. Preferably, the CTM-comprising polypeptide specifically binding to GPD1 binds to GPD1-like protein with a K_{D} value at least 10 fold, preferably at least 20 fold, more preferably at least 50 fold, most preferably at least 100 fold higher than the K_{D} value of said CTM-comprising polypeptide for GPD1. More preferably, the CTM-comprising polypeptide specifically binding to GPD1 does not detectably bind to GPD1-like protein. As will be understood by the skilled person, in case the indirect GPD1 inhibitor is a CTM-comprising polypeptide, said CTM-comprising polypeptide does not have to be, but may be, a direct inhibitor of GPD1. Thus, preferably, the CTM-comprising polypeptide is a direct inhibitor of GPD1.

More preferably, the indirect GPD1 inhibitor is an antisense oligonucleotide. The term “antisense oligonucleotide” is known to the skilled person and relates to an oligonucleotide hybridizing to a target RNA, causing the formation of a DNA/RNA hybrid. Said DNA/RNA hybrid is a substrate for RNase H, which degrades the RNA portion of said DNA/RNA hybrid. Thus, the antisense oligonucleotide comprises at least five, preferably at least seven, more preferably at least nine, or, most preferably, at least ten DNA nucleotides. Preferably, the antisense oligonucleotide has a length of at least 15 nucleotides, preferably at least 18 nucleotides, still more preferably at least 20 nucleotides.

Most preferably, the indirect GPD1 inhibitor is a polynucleotide inducing RNA interference. As used herein, “RNA interference (RNAi)” refers to sequence-specific, post-transcriptional gene silencing of a selected target gene by degradation of RNA transcribed from the target gene (target RNA). Target RNAs, preferably, are GPD1 RNAs, i.e. RNAs transcribed from a GPD1 gene as specified above. It is to be understood that silencing as used herein does not necessarily mean the complete abolishment of expression. RNAi, preferably, reduces expression by at least 40%, more preferably at least 60%, even more preferably at least 80%, most preferably at least 90% as compared to the expression level in a reference without RNAi.

RNAi requires in the cell the presence of dsRNAs that are homologous in sequence to the target RNAs. The term “dsRNA” refers to RNA having a duplex structure comprising two complementary and anti-parallel nucleic acid strands. The RNA strands forming the dsRNA may have the same or a different number of nucleotides, whereby one of the strands of the dsRNA can be the target RNA. It is, however, also contemplated by the present invention that the dsRNA is formed between two sequence stretches on the same RNA molecule.

RNAi may be used to specifically inhibit expression of the target RNAs of the present invention in vivo. Accordingly, it may be used for the medical uses as specified elsewhere herein. For such therapeutic approaches, expression constructs for siRNA may be introduced into cancer cells of the host. Accordingly, siRNA may be combined efficiently with other therapy approaches. Methods relating to the use of RNAi to silence genes in animals, including mammals, are known in the art (see, for example, Hammond et al. (2001), Nature Rev. Genet. 2, 110-119; Bernstein et al. (2001), Nature 409, 363-366; WO 9932619; and Elbashir et al. (2001), Nature 411: 494-498).

Thus, according to the present invention, the indirect inhibitor of GPD1, preferably is an RNAi agent. As used herein, the term “RNAi agent” refers to an shRNA, a siRNA agent, or a miRNA agent as specified below. The RNAi agent of the present invention is of sufficient length and complementarity to stably interact with the target RNA, i.e. it comprises at least 15, at least 17, at least 19, at least 21, at least 22 nucleotides complementary to the target RNA. By “stably interact” is meant interaction of the RNAi agent or its products produced by the cell with a target RNA, e.g., by forming hydrogen bonds with complementary nucleotides in the target RNA under physiological conditions.

The term “siRNA agent” as meant herein encompasses: a) a dsRNA consisting of at least 15, at least 17, at least 19, at least 21 consecutive nucleotides base-paired, i.e. forming hydrogen bonds with complementary nucleotides. b) a small interfering RNA (siRNA) molecule or a molecule comprising an siRNA molecule. The siRNA is a single-stranded RNA molecule with a length, preferably, greater than or equal to 15 nucleotides and, preferably, a length of 15 to 49 nucleotides, more preferably 17 to 30 nucleotides, and most preferably 17 to 30 nucleotides, preferably 17, 18, 19, 20, 21, 22, 23, 24, 25, 26, 27, 28, 29, or 30 nucleotides. According to the present invention, the term “molecule comprising a siRNA molecule” includes RNA molecules from which a siRNA is processed by a cell, preferably by a mammalian cell. Thus, a molecule comprising a siRNA molecule, preferably, is a small hairpin RNA, also known as shRNA. As used herein, the term “shRNA” relates to a, preferably artificial, RNA molecule forming a stem-loop structure comprising at least 10, preferably at least 15, more preferably at least 17, most preferably at least 20 nucleotides base-paired to a complementary sequence on the same mRNA molecule (“stem”), i.e. as a dsRNA, separated by a stretch of non-base-paired nucleotides (“loop”). c) a polynucleotide encoding a) or b), wherein, preferably, said polynucleotide is operatively linked to an expression control sequence. Thus, the function of the siRNA agent to inhibit expression of the target gene can be modulated by said expression control sequence. Preferred expression control sequences are those, which can be regulated by exogenous stimuli, e.g. the tet operator, whose activity can be regulated by tetracycline, or heat inducible promoters. Alternatively or in addition, one or more expression control sequences can be used which allow tissue-specific expression of the siRNA agent.

It is, however, also contemplated by the current invention that the RNAi agent is a miRNA agent. A “miRNA agent” as meant herein encompasses: a) a pre-microRNA, i.e. a mRNA comprising at least 30, at least 40, at least 50, at least 60, at least 70 nucleotides base-paired to a complementary sequence on the same mRNA molecule (“stem”), i.e. as a dsRNA, separated by a stretch of non-base-paired nucleotides (“loop”). b) a pre-microRNA, i.e. a dsRNA molecule comprising a stretch of at least 19, at least 20, at least 21, at least 22, at least 23, at least 24, at least 25 base-paired nucleotides formed by nucleotides of the same RNA molecule (stem), separated by a loop. c) a microRNA (miRNA), i.e. a dsRNA comprising at least 15, at least 17, at least 18, at least 19, at least 21 nucleotides on two separate RNA strands. d) a polynucleotide encoding a) or b), wherein, preferably, said polynucleotide is operatively linked to an expression control sequence as specified above. Preferably, the miRNA comprises, more preferably consists of, the nucleotide sequence of SEQ ID NO:1.

Also preferably, the indirect inhibitor of GPD1 comprises two CRISPR/Cas oligonucleotides. The CRISPR/Cas system has been known for several years as a convenient system for inducing knock-out mutations, i.e. deletions, preferably of chromosomal genes. The skilled person knows how to design appropriate oligonucleotides, which are, preferably, expressed from a vector, to induce deletion of a DNA sequence of interest. Preferably, said deletion is a partial deletion, more preferably deletion of a portion of the gene essential for function; most preferably said deletion is a complete deletion of at least the whole coding region. Preferably, the CRISPR/Cas oligonucleotides comprise at least one, preferably both of SEQ ID NOs 2 and 3; more preferably, the CRISPR/Cas oligonucleotides comprise an oligonucleotide having SEQ ID NO: 4.

As used herein, the term “polypeptide variant” relates to any chemical molecule comprising at least one polypeptide as specified above, having the indicated activity, but differing in structure from said polypeptide indicated above. Preferably, the polypeptide variant comprises a peptide having an amino acid sequence corresponding to an amino acid sequence of 5 to 200, more preferably 6 to 100, even more preferably 7 to 50, or, most preferably, 8 to 30 consecutive amino acids comprised in a polypeptide as specified above. Moreover, also encompassed are further polypeptide variants of the aforementioned polypeptides. Such polypeptide variants have at least the same essential biological activity as the specific polypeptides. Moreover, it is to be understood that a polypeptide variant as referred to in accordance with the present invention shall have an amino acid sequence which differs due to at least one amino acid substitution, deletion and/or addition, wherein the amino acid sequence of the variant is still, preferably, at least 50%, 60%, 70%, 80%, 85%, 90%, 92%, 95%, 97%, 98%, or 99% identical with the amino acid sequence of the specific polypeptide. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the sequence it is compared to for optimal alignment. The percentage is calculated by determining, preferably over the whole length of the peptide, the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Polypeptide variants referred to above may be allelic variants or any other species specific homologs, paralogs, or orthologs. Moreover, the polypeptide variants referred to herein include fragments of the specific polypeptides or the aforementioned types of polypeptide variants as long as these fragments and/or variants have the biological activity as referred to above. Such fragments may be or be derived from, e.g., degradation products or splice variants of the polypeptides. Further included are variants, which differ due to posttranslational modifications such as e.g. phosphorylation, glycosylation, ubiquitinylation, sumoylation, or myristylation, by including non-natural amino acids, and/or by being peptidomimetics.

The term “polynucleotide variant”, as used herein, relates to a variant of a polynucleotide related to herein comprising a nucleic acid sequence characterized in that the sequence can be derived from the aforementioned specific nucleic acid sequence by at least one nucleotide substitution, addition and/or deletion, wherein the polynucleotide variant shall have the activity as specified for the specific polynucleotide. Moreover, it is to be understood that a polynucleotide variant as referred to in accordance with the present invention shall have a nucleic acid sequence, which differs due to at least one nucleotide substitution, deletion and/or addition. Preferably, said polynucleotide variant is an ortholog, a paralog or another homolog of the specific polynucleotide. Also preferably, said polynucleotide variant is a naturally occurring allele of the specific polynucleotide. Polynucleotide variants also encompass polynucleotides comprising a nucleic acid sequence, which is capable of hybridizing to the aforementioned specific polynucleotides, preferably, under stringent hybridization conditions. These stringent conditions are known to the skilled worker and can be found in Current Protocols in Molecular Biology, John Wiley & Sons, N. Y. (1989), 6.3.1-6.3.6. A preferred example for stringent hybridization conditions are hybridization conditions in 6× sodium chloride/sodium citrate (═SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1× to 5×SSC (pH 7.2). If organic solvent is present in the abovementioned buffer, for example 50% formamide, the temperature under standard conditions is approximately 42° C. The hybridization conditions for DNA:DNA hybrids are preferably, for example, 0.1×SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined, for example, for a nucleic acid with approximately 100 bp (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, polynucleotide variants are obtainable by PCR-based techniques such as mixed oligonucleotide primer-based amplification of DNA, i.e. using degenerated primers against conserved domains of a polypeptide of the present invention. Conserved domains of a polypeptide may be identified by a sequence comparison of the nucleic acid sequence of the polynucleotide or the amino acid sequence of the polypeptide of the present invention with sequences of other organisms. As a template, DNA or cDNA from bacteria, fungi, plants or, preferably, from animals may be used. Further, variants include polynucleotides comprising nucleic acid sequences which are at least 70%, at least 75%, at least 80%, at least 85%, at least 90%, at least 95%, at least 98% or at least 99% identical to the specifically indicated nucleic acid sequences. Moreover, also encompassed are polynucleotides which comprise nucleic acid sequences encoding amino acid sequences which are at least 70%, at least 75%, at least 80%, at least 85%, at least 90%, at least 95%, at least 98% or at least 99% identical to the amino acid sequences specifically indicated. The percent identity values are, preferably, calculated over the entire amino acid or nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution. 25, 351-360, 1987, Higgins et al., CABIOS, 5 1989: 151-153) or the programs Gap and BestFit (Needleman and Wunsch (J. Mol. Biol. 48; 443-453 (1970)) and Smith and Waterman (Adv. Appl. Math. 2; 482-489 (1981))), which are part of the GCG software packet (Genetics Computer Group, 575 Science Drive, Madison, Wis., USA 53711 (1991)), are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

A polynucleotide comprising a fragment of any of the specifically indicated nucleic acid sequences is also encompassed as a variant polynucleotide of the present invention. The fragment shall still encode a polypeptide, which still has the activity as specified. Accordingly, the polypeptide encoded may comprise or consist of the domains of the inhibitory polypeptide of the present invention conferring the said biological activity. A fragment as meant herein, preferably, comprises at least 50, at least 100, at least 250 or at least 500 consecutive nucleotides of any one of the specific nucleic acid sequences or encodes an amino acid sequence comprising at least 20, at least 30, at least 50, at least 80, at least 100 or at least 150 consecutive amino acids of any one of the specific amino acid sequences.

The polynucleotides of the present invention either consist, essentially consist of, or comprise the aforementioned nucleic acid sequences. Thus, they may contain further nucleic acid sequences as well. Specifically, the polynucleotides of the present invention may encode fusion proteins wherein one partner of the fusion protein is a polypeptide being encoded by a nucleic acid sequence recited above. Such fusion proteins may comprise as additional part polypeptides for monitoring expression (e.g., green, yellow, blue or red fluorescent proteins, alkaline phosphatase and the like) or so called “tags” which may serve as a detectable marker or as an auxiliary measure for purification purposes. Tags for the different purposes are well known in the art and are described elsewhere herein.

The polynucleotide of the present invention shall be provided, preferably, either as an isolated polynucleotide (i.e. isolated from its natural context) or in genetically modified form. The polynucleotide, preferably, is DNA, including cDNA, or RNA. The term encompasses single as well as double stranded polynucleotides. Moreover, preferably, comprised are also chemically modified polynucleotides including naturally occurring modified polynucleotides such as glycosylated or methylated polynucleotides or artificial modified one such as biotinylated polynucleotides.

The term “treating” refers to ameliorating the diseases or disorders referred to herein or the symptoms accompanied therewith to a significant extent. Said treating as used herein also includes an entire restoration of the health with respect to the diseases or disorders referred to herein. It is to be understood that treating as used in accordance with the present invention may not be effective in all subjects to be treated. However, the term shall preferably require that a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment is effective for at least 60%, at least 70%, at least 80%, or at least 90% of the subjects of a given cohort or population.

The term “preventing” refers to retaining health with respect to the diseases or disorders referred to herein for a certain period of time in a subject. It will be understood that the said period of time is dependent on the amount of the drug compound, which has been administered and individual factors of the subject discussed elsewhere in this specification. It is to be understood that prevention may not be effective in all subjects treated with the compound according to the present invention. However, the term preferably requires that a statistically significant portion of subjects of a cohort or population is effectively prevented from suffering from a disease or disorder referred to herein or its accompanying symptoms. Preferably, a cohort or population of subjects is envisaged in this context, which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification.

According to the present invention, treating and preventing comprise administration of an inhibitor of GPD1, preferably at an effective dose as specified elsewhere herein. Preferably, treating and/or preventing cancer includes treating and/or preventing cancer relapse and/or metastasis. Preferably, treating and/or preventing cancer comprises neoadjuvant administration of said GPD1 inhibitor, i.e. administration of at least one inhibitor of GPD1 before a main treatment is administered, wherein said main treatment, preferably, is surgery, preferably potentially curative or curative surgery, or is radiotherapy. Preferably, treating and/or preventing cancer relapse and/or metastasis is preventing cancer relapse or is preventing metastasis. Preferably, treating and/or preventing comprises administration of at least one cancer therapeutic agent.

As used herein, the term “cancer therapy” relates to a medical measure administered with the intention to treat cancer. The term, preferably, relates to administration to a subject of a chemical compound or of a further medical measure known to inhibit growth of cancer cells, to kill cancer cells, to remove cancer cells, or to cause the body of a patient to inhibit the growth of or to kill cancer cells. The term “administration of a chemical compound”, preferably, includes administration of an agent releasing in the body of a subject such chemical compound having the aforementioned effect, i.e., more preferably, comprises administration of a prodrug. The term “further medical measure”, as used herein, preferably, relates to a medical measure used in the treatment of cancer, but not comprising administration of a chemical compound or its prodrug to a subject; thus the term preferably relates to surgery and/or radiotherapy. More preferably, cancer therapy comprises administration of a further cancer therapeutic agent as specified elsewhere herein, preferably of a chemotherapeutic agent, of an agent for targeted therapy, of an immunotherapeutic agent, of a biotherapeutic agent, and/or of a virotherapeutic agent, or any combination thereof. Preferably, cancer therapy, in particular chemotherapy, relates to a complete cycle of treatment, i.e. a series of several (e.g. four, six, or eight) doses of antineoplastic drug or drugs administered to a subject separated by several days or weeks without such application.

In accordance with the above, the term “further cancer therapeutic agent” relates to at least one chemical compound used in cancer therapy. The term, preferably, relates to a chemical compound known to inhibit growth of cancer cells, to kill cancer cells, to remove cancer cells, or to cause the body of a patient to inhibit the growth of or to kill cancer cells in the treatment of cancer by application of said chemical substance to a patient in need thereof; or by administration of an agent releasing such chemical substance in the body of a subject, i.e., more preferably, by administration of a prodrug. More preferably, the further cancer therapeutic agent is a chemotherapeutic agent, an agent for targeted therapy, an agent for immunotherapy, a biotherapeutic agent, or a virotherapeutic agent, or any combination thereof.

It is to be understood that the term “further cancer therapeutic agent”, as used herein, does not relate to an inhibitor of GPD1, despite the fact that, as specified elsewhere herein, an inhibitor of GPD1 has the property of being a cancer therapeutic agent; thus, as used herein, the inhibitor of GPD1 is not a chemotherapeutic agent, an agent for targeted therapy, an agent for immunotherapy, a biotherapeutic agent, or a virotherapeutic agent; thus, preferably, a chemotherapeutic agent, an agent for targeted therapy, an agent for immunotherapy, a biotherapeutic agent, or a virotherapeutic agent according to the present invention, preferably, does not have the property of inhibiting GPD1

As used herein, the term “chemotherapy” relates to treatment of a subject with an antineoplastic agent. Preferably, chemotherapy is a treatment including administration of at least one of alkylating agents (e.g. cyclophosphamide), platinum (e.g. carboplatin), anthracyclines (e.g. doxorubicin, epirubicin, idarubicin, or daunorubicin), topoisomerase II inhibitors (e.g. etoposide, irinotecan, topotecan, camptothecin, or VP16), anaplastic lymphoma kinase (ALK)-inhibitors (e.g. Crizotinib or AP26130), aurora kinase inhibitors (e.g. N-[4-[4-(4-Methylpiperazin-1-yl)-6-[(5-methyl-1H-pyrazol-3-yl)amino]pyrimidin-2-yl]sulfanylphenyl]cyclopropanecarboxamide (VX-680)), antiangiogenic agents (e.g. Bevacizumab), Iodine131-1-(3-iodobenzyl)guanidine (therapeutic metaiodobenzylguanidine), and HDAC8-Inhibitors, alone or any suitable combination thereof.

The term “targeted therapy”, as used herein, relates to application to a patient of a chemical substance which, for example, is known to block growth of cancer cells by interfering with specific molecules known to be necessary for tumorigenesis or cancer or cancer cell growth. Examples known to the skilled artisan are small molecules like, e.g. Bcl-2-Inhibitors (e.g. Obatoclax) and PARP-inhibitors (e.g. Iniparib), or monoclonal antibodies like, e.g., Rituximab or Trastuzumab.

The term “immunotherapy” as used herein relates to the treatment of cancer by modulation of the immune response of a subject. Said modulation may be inducing, enhancing, or suppressing said immune response.

A “biotherapeutic agent” as used herein relates to a biological oligo- or polymeric molecule, comprising e.g. amino acids, nucleotides, lipids and/or saccharides, said molecule being destructive to malignant cells and tissues. Examples of biotherapeutic agents include inhibitory antibodies and fragments thereof, agonists and antagonists of intra- or extracellular receptors, ribozymes, siRNAs, peptide and nucleic acid aptamers or dominant negative derivatives of cellular effectors.

The term “virotherapeutic agent”, as used herein, relates to a virus or to a biotherapeutic agent derived from a virus, said virus or molecule being destructive to malignant cells or tissues. Said virus may be a wildtype virus or a genetically modified virus. Examples of viruses that can be used as virotherapeutic agents are parvovirus H1, measles virus, or foamy virus; genetically modified viruses include retroviruses like HIV, adenovirus, herpesviruses, and the like. Examples for virus-derived molecules are the herpesviral thymidine-kinase, the parvovirus NS1 cytotoxin, the apoptin protein derived from chicken anemia virus, mengovirus- or poliovirus-derived RNA replicons, and Semliki Forest virus LacZ particles.

“Cancer” in the context of this invention refers to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body (“metastasis”). Moreover, cancer may entail recurrence of cancer cells after an initial treatment apparently removing cancer cells from a subject (“relapse”).

Preferably, the cancer is selected from the list consisting of acute lymphoblastic leukemia, acute myeloid leukemia, adrenocortical carcinoma, aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, chronic lymphocytic leukemia, chronic myelogenous leukemia, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, gallbladder cancer, gastric cancer, gastrointestinal stromal tumor, gestational trophoblastic tumor, hairy cell leukemia, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, medulloepithelioma, melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sézary syndrome, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, waldenstrom macroglobulinemia, and wilms tumor. More preferably, the cancer is brain cancer or bladder cancer. Preferably, the brain cancer is a glioma, more preferably an astrocytoma, or an oligodendroglioma, or an oligoastrocytoma, most preferably a glioblastoma multiforme.

The term “subject”, as used herein, preferably relates to a higher multicellular animal, preferably a vertebrate. More preferably, the subject is a mammal, even more preferably a rat, mouse, or human, most preferably, the subject is a human. Preferably, the subject comprises GPD1, preferably human GPD1, physiologically and/or recombinantly expressed. More preferably, the subject comprises GPD1 physiologically.

Advantageously, it was found in the work underlying the present invention that GPD1 is overproduced in tumor cells and in tumor stem cells, in particular brain tumor stem cells, but not in normal tissue stem cells, in particular not in neural stem cells. Moreover, it was found that knockdown of GPD1 expression in tumor cells promotes cell differentiation and prolongs in vivo survival in a mouse brain tumor model.

The definitions made above apply mutatis mutandis to the following. Additional definitions and explanations made further below also apply for all embodiments described in this specification mutatis mutandis.

The present invention further relates to a medicament for treating and/or preventing cancer comprising (i) a GPD1 inhibitor and (ii) a pharmaceutically acceptable carrier.

The terms “pharmaceutical composition” and “medicament”, as used herein, relate to a composition comprising the compounds of the present invention in a pharmaceutically acceptable form and a pharmaceutically acceptable carrier. The compounds of the present invention can be formulated as pharmaceutically acceptable salts. Acceptable salts comprise acetate, methylester, HCl, sulfate, chloride and the like. The pharmaceutical compositions are, preferably, administered topically or systemically. Suitable routes of administration conventionally used for drug administration are oral, intravenous, or parenteral administration as well as inhalation. However, depending on the nature and mode of action of a compound, the pharmaceutical compositions may be administered by other routes as well. For example, polynucleotide compounds may be administered in a gene therapy approach by using viral vectors, viruses or liposomes. Moreover, the compounds can be administered in combination with other drugs either in a common pharmaceutical composition or as separated pharmaceutical compositions wherein said separated pharmaceutical compositions may be provided in form of a kit of parts.

The compounds are, preferably, administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables.

The carrier(s) must be acceptable in the sense of being compatible with the other ingredients of the formulation and being not deleterious to the recipient thereof. The pharmaceutical carrier employed may be, for example, a solid, a gel or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa.

The diluent(s) is/are preferably selected so as not to affect the biological activity of the inhibitor of GPD1 and potential further pharmaceutically active ingredients. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

A therapeutically effective dose refers to an amount of the compounds to be used in a pharmaceutical composition of the present invention, which prevents, ameliorates or treats a condition referred to herein. Therapeutic efficacy and toxicity of compounds can be determined by standard pharmaceutical procedures in cell culture or in experimental animals, e.g., by determining the ED50 (the dose therapeutically effective in 50% of the population) and/or the LD50 (the dose lethal to 50% of the population). The dose ratio between therapeutic and toxic effects is the therapeutic index, and it can be expressed as the ratio, LD50/ED50.

The dosage regimen will be determined by the attending physician, preferably taking into account relevant clinical factors and, preferably, in accordance with any one of the methods described elsewhere herein. As is well known in the medical arts, a dosage for any one patient may depend upon many factors, including the patient's size, body surface area, age, the particular compound to be administered, sex, time and route of administration, general health, and other drugs being administered concurrently. Progress can be monitored by periodic assessment. A typical dose can be, for example, in the range of 1 μg to 10000 μg, preferably per day; however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per hour, respectively. However, depending on the subject and the mode of administration, the quantity of substance administration may vary over a wide range to provide from about 0.01 mg per kg body mass to about 10 mg per kg body mass, preferably. The pharmaceutical compositions and formulations referred to herein are administered at least once in order to treat or prevent a disease or condition recited in this specification. However, the said pharmaceutical compositions may be administered more than one time, for example from one to four times daily up to a non-limited number of days.

Specific pharmaceutical compositions are prepared in a manner well known in the pharmaceutical art and comprise at least an inhibitor of GPD1 as an active compound in admixture or otherwise associated with a pharmaceutically acceptable carrier or diluent. For making those specific pharmaceutical compositions, the active compound(s) will usually be mixed with a carrier or the diluent, or enclosed or encapsulated in a capsule, sachet, cachet, paper or other suitable containers or vehicles. The resulting formulations are to be adapted to the mode of administration, i.e. in the forms of tablets, capsules, suppositories, solutions, suspensions or the like. Dosage recommendations shall be indicated in the prescribers or user instructions in order to anticipate dose adjustments depending on the considered recipient.

The present invention also relates to a kit comprising a GPD1 inhibitor, preferably formulated in a pharmaceutically acceptable carrier, comprised in a housing.

The term “kit”, as used herein, refers to a collection of the aforementioned compounds, means or reagents of the present invention, which may or may not be packaged together. Components of the kit, preferably the inhibitor of GPD1 and a pharmaceutically acceptable carrier, may be comprised by separate vials (i.e. as a kit of separate parts) or provided in a single vial. Moreover, it is to be understood that the kit of the present invention is preferably to be used for practicing the methods referred to elsewhere herein. It is, preferably, envisaged that all components are provided in a ready-to-use manner for practicing the methods referred to elsewhere herein. Further, the kit preferably contains instructions for carrying out the said methods. The instructions can be provided by a user's manual in paper form or electronic form. For example, the manual may comprise instructions for interpreting the results obtained when carrying out the aforementioned methods using the kit of the present invention.

Preferably, the kit further comprises a means of administration for said inhibitor of GPD1. Preferred means for administering medicaments are well known in the art. Preferably, said means comprises a delivery unit for the administration of the compound or medicament and a storage unit for storing said compound or medicament until administration. However, it is also contemplated that the means of the current invention may appear as separate means in such an embodiment and are, preferably, packaged together as a kit. Preferred means are those, which can be applied without the particular knowledge of a specialized technician. Preferably, the means of administration is a syringe, more preferably with a needle, comprising the compound or medicament of the invention. In another preferred embodiment, the means of administration is an intravenous infusion (IV) equipment, preferably an IV bag or an IV bottle, comprising the compound or medicament. Also preferably, the means of administration is an endoscopic device comprising the compound or medicament for flushing a site of tumor resection before and/or after surgical resection of a tumor. In another preferred embodiment, the means of administration is an inhaler comprising the compound of the present invention, wherein, more preferably, said compound is formulated for administration as an aerosol.

More preferably, the kit further comprises a means for dissolving and/or adjusting the concentration of the inhibitor of GPD1. A preferred means for dissolving and/or adjusting the concentration of the inhibitor of GPD1 is a pharmaceutically acceptable carrier, e.g., more preferably, water, Ringer's solution, phosphate-buffered saline, or another pharmaceutically acceptable carrier as specified elsewhere herein. Also preferably, the kit further comprises a further cancer therapeutic agent, more preferably a chemotherapeutic agent, an agent for targeted therapy, an agent for immunotherapy, a biotherapeutic agent, a virotherapeutic agent, or any combination thereof.

The present invention also relates to a method for identifying a subject suffering from cancer susceptible to cancer treatment by administration of a GPD1 inhibitor comprising

a) determining in a sample of said subject the amount of a GPD1 gene product,
b) comparing said amount determined in step a) to a reference, and
c) based on the result of step b), identifying a subject susceptible to cancer treatment by administration of a GPD1 inhibitor.

The method for identifying a subject of the present invention, preferably, is an in vitro method. Moreover, it may comprise steps in addition to those explicitly mentioned above. For example, further steps may relate, e.g., to obtaining a sample for step a), or comparing a result from step a) to a reference value in step b). Moreover, one or more of said steps may be performed by automated equipment.

The term “identifying a subject susceptible to treatment”, as used herein, relates to identifying a subject having an increased probability of responding to said treatment. Preferably, the term “responding to a treatment” relates to at least showing an amelioration of the symptoms associated with the disease treated. In the case of cancer treatment or prevention, preferably, responding to treatment is showing a, preferably significant, reduction of the size of a primary tumor; a, preferably significant, reduction of the number and/or the size of metastases of a primary tumor; and/or at least a, preferably significant, reduction in size of relapse, or prevention of relapse and/or increase in progression free survival Preferably, a subject is identified as being susceptible to treatment by GPD1 inhibitor administration if the amount of GPD1 in a sample of said subject is increased compared to a sample from an apparently healthy reference subject or to a reference of healthy tissue. Conversely, a subject is identified as being susceptible to treatment by GPD1 inhibitor administration if the amount of GPD1 in a sample of said subject is equal to or increased compared to a sample from a reference subject known to suffer from a cancer susceptible to cancer treatment by administration of a GPD1 inhibitor.

References for a marker as set forth herein in the context of the present invention can be easily established. Moreover, an amount of a marker in a test sample from a subject can simply be compared to the reference amount, respectively. It is understood by the skilled person that comparing may be comparing values actually measured, e.g. a relative or absolute GPD1 concentration, to a reference value. However, the skilled person also knows mathematical, in particular statistical, methods of transforming such measured values into derived values, e.g. into a response index.

Preferably, a reference is obtained from a sample from (i) at least one subject known to be susceptible to treating and/or preventing cancer, cancer relapse, and/or metastasis by GPD1 inhibitor administration, from (ii) at least one subject known not to be susceptible to treating and/or preventing cancer, cancer relapse, and/or metastasis by GPD1 inhibitor administration, from (iii) both at least one subject known to be susceptible to treating and/or preventing cancer, cancer relapse, and/or metastasis by GPD1 inhibitor administration and at least one subject known not to be susceptible to said treatment, or from (iv) at least one apparently healthy subject. The reference can also be or be derived from the average or mean obtained from a group of such samples. How to calculate a suitable reference, preferably a reference value, a reference ratio, or a reference index, is well known in the art. The population of subjects referred to before shall comprise a plurality of subjects, preferably, at least 5, 10, 50, 100, 1,000 or 10,000 subjects. It is to be understood that the subject to be assessed by the method of the present invention and the subjects of the said plurality of subjects preferably are of the same species.

The sensitivity and specificity of a diagnostic test depends on more than just the analytical “quality” of the test; they also depend on the definition of what constitutes an abnormal result. In practice various methods of statistically evaluating measurement results, including comparison to references, are available and known to the skilled person. As an example, preferably, Receiver Operating Characteristic curves, or “ROC” curves, are typically calculated by plotting the value of a variable versus its relative frequency in a population at high risk of relapse and/or metastasis and a population at low or no risk of relapse and/or metastasis. For any particular marker, a distribution of marker levels for subjects will likely overlap. Under such conditions, a test does not absolutely distinguish patients with responding to treatment from patients not responding to treatment with 100% accuracy, and the area of overlap indicates where the test cannot distinguish patients. A threshold is selected, above which the test is considered as indicating a patient responding to treatment and below which the test is considered as indicating a patient not responding to treatment. The area under the ROC curve is a measure of the probability that the perceived measurement will allow correct diagnosis of a subject. These methods are well known in the art. See, e.g., Hanley et al, Radiology 143: 29-36 (1982).

In certain embodiments, a reference is selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%.

The terms “sample” and “test sample”, as used herein, refer to a sample of a body fluid, to a sample of separated cells or to a sample from a tissue or an organ or to a sample of wash/rinse fluid obtained from an outer or inner body surface. Also included as a sample is a sample of cultured cells. As used herein, the term “body fluid” preferably relates to all bodily fluids of a subject known to comprise or suspected to comprise GPD1, including blood, plasma, lacrimal fluid, urine, lymph, cerebrospinal fluid, bile, stool, sweat, and saliva. More preferably, the body fluid is blood, serum, or plasma. Samples of body fluids can be obtained by well known techniques including, e.g., venous or arterial puncture, epidermal puncture, and the like. Preferably, the sample is a sample suspected to comprise a tumor cells and/or a tumor stem cell; more preferably the sample is a tumor sample.

As referred to herein, a “tumor stem cell”, also known as “cancer stem cell”, is known to the skilled person as cancer cell having the propensity for self-renewal and/or for giving rise to cancer cell types present in a tumor. Preferably, the tumor stem cells are brain tumor stem cells. As will be understood by the skilled person, tumor stem cells are different from normal stem cells; thus, in particular, brain tumor stem cells are not neural stem cells.

The term “gene product” as used herein, preferably, relates to a transcript, and thus to mRNA, or to a polypeptide produced by a cell. Thus, the GPD1 gene product, preferably, is a RNA, preferably a mRNA, transcribed from a GPD1 gene, preferably the human GPD1 gene. Also preferably, the GPD1 gene product is a GPD1 polypeptide, preferably a human GPD1, translated from a GPD1 mRNA, preferably human GPD1 mRNA. As used herein, the term gene product includes splice variants of mRNAs and polypeptides translated from such splice variants.

The present invention further relates to a use of a GPD1 inhibitor for the manufacture of a medicament for use in treating and/or preventing cancer.

The present invention further relates to a method for treating and/or preventing cancer in a subject, comprising administering to said subject a GPD1 inhibitor.

Furthermore, the present invention relates to a method for identifying a compound for treating and/or preventing cancer, comprising

a) contacting a reaction mixture comprising a GPD1 polypeptide, glycerol-3-phosphate, and nicotine-adenine-dinucleotide (NAD) with a compound suspected to have the activity of being a direct GPD1 inhibitor,
b) further contacting said reaction mixture of a) with a luciferase and a luciferase substrate,
c) determining (i) NAD(H) absorbance and/or (ii) the luciferase-mediated luminescence in the reaction mixture, and
d) identifying a compound for treating and/or preventing cancer based on the result of the determination step of c).

The method for identifying a compound of the present invention, preferably, is an in vitro method. Moreover, it may comprise steps in addition to those explicitly mentioned above. Moreover, one or more of said steps may be performed by automated equipment.

The terms “nicotine-adenine-dinucleotide” and “NAD” are known to the skilled person to relate to a redox coenzyme (CAS-No: 53-84-9). The term “luciferase” (EC 1.13.12.5, 1.13.12.6, 1.13.12.7, 1.13.12.8, 1.13.12.13, or 1.13.12.18) is also known to the skilled person as relating to a group of oxygenases producing bioluminescence from NADH as a coenzyme, i.e. from the reduced form of NAD. As is known to the skilled person, the luciferase substrate is specific for a particular luciferase, e.g. the substrate of Renilla-luciferin 2-monooxygenase (Renilla luciferase, EC 1.13.12.5) is coelenterazine (CAS No. 55779-48-1). Thus, preferably, the luciferase is Renilla-luciferin 2-monooxygenase and, preferably, the luciferase substrate is coelenterazine. Preferably, the step of contacting the reaction mixture with a luciferase is performed at the optimal pH of said luciferase; more preferably, said step of contacting the reaction mixture with a luciferase is performed at a pH of from 8 to 10, preferably a pH of 9±0.5, more preferably a pH of 9±0.25. Methods for determining bioluminescence from a luciferase reaction are known in the art.

Methods of determining NADH absorbance are also known in the art; preferably, the concentration of NADH is determined by photometric determining NADH absorbance at a wavelength in the range of from 320 nm to 360 nm, preferably of from 330 to 350 nm, more preferably at 340 nm±5 nm, most preferably at 340 nm. As is understood by the skilled person, NADH has an absorbance peak at the aforesaid wavelength, whereas NAD does not; thus, by measuring absorbance in the aforesaid range of wavelengths, the concentration of NADH can be determined. Preferably, a known amount of NAD is included into the reaction mixture, enabling calculation of the NAD/NADH ratio. Preferably, in the method for identifying a compound for treating and/or preventing cancer of the present invention, NADH absorbance and luciferase-mediated luminescence in the reaction mixture are determined; more preferably, in the method for identifying a compound for treating and/or preventing cancer of the present invention, NADH absorbance and luciferase-mediated luminescence in the reaction mixture are determined successively, wherein, even more preferably, NADH absorbance is determined first.

Preferably, a decreased concentration of NADH in the reaction mixture in the presence of a compound suspected to have the activity of being a direct GPD1 inhibitor compared to a reaction mixture not comprising said compound is indicative of said compound being a direct GPD1 inhibitor; also preferably, a decreased luciferase-mediated luminescence in the reaction mixture in the presence of a compound suspected to have the activity of being a direct GPD1 inhibitor compared to a reaction mixture not comprising said compound is indicative of said compound being a direct GPD1 inhibitor. More preferably, a decreased concentration of NADH and a decreased luciferase-mediated luminescence in the reaction mixture in the presence of a compound suspected to have the activity of being a direct GPD1 inhibitor compared to a reaction mixture not comprising said compound are indicative of said compound being a direct GPD1 inhibitor. Thus, preferably, a compound is identified as a direct GPD1 inhibitor if both a decreased concentration of NADH and a decreased luciferase-mediated luminescence in the reaction mixture are determined in the method for identifying a compound for treating and/or preventing cancer.

In view of the above, the following embodiments are preferred:

1. An inhibitor of glycerol-3-phosphate dehydrogenase 1 (GPD1 inhibitor) for use in treatment and/or prevention of cancer.
2. The GPD1 inhibitor for use of embodiment 1, wherein said cancer is brain cancer or bladder cancer.
3. The GPD1 inhibitor for use of embodiment 1 or 2, wherein said brain cancer is a glioma, more preferably an astrocytoma, an oligodendroglioma or an oligoastocytoma, most preferably a glioblastoma multiforme.
4. The GPD1 inhibitor for use of any one of embodiments 1 to 3, wherein said treatment of cancer further comprises administration of a cancer therapeutic agent, preferably comprises administration of chemotherapy.
5. The GPD1 inhibitor for use of any one of embodiments 1 to 4, wherein said GPD1 inhibitor is a direct GPD1 inhibitor.
6. The GPD1 inhibitor for use of embodiment 5, wherein said direct GPD1 inhibitor is a polypeptide or polynucleotide specifically binding and inhibiting GPD1, preferably selected from the list consisting of an antibody, an aptamer, an anticalin, and a Designed Ankyrin Repeat Protein (DARPin).
7. The GPD1 inhibitor for use of any one of embodiments 1 to 4, wherein said GPD1 inhibitor is an indirect GPD1 inhibitor.
8. The GPD1 inhibitor for use of embodiment 7, wherein said indirect GPD1 inhibitor is a polypeptide comprising a lysosome-degradation sequence, preferably a chaperone-mediated autophagy-targeting motif (CTM).
9. The GPD1 inhibitor for use of embodiment 7, wherein said indirect GPD1 inhibitor is a polynucleotide.
10. The GPD1 inhibitor for use of embodiment 9, wherein said indirect inhibitor specifically binds to a polynucleotide encoding GPD1 and is selected from the group consisting of a shRNA, a siRNA, a miRNA agent, an antisense molecule, an antisense oligonucleotide, a ribozyme, and a pair of CRISPR/Cas oligonucleotides.
11. The GPD1 inhibitor for use of embodiment 9 or 10, wherein said inhibitor comprises at least one nucleic acid comprising at least one nucleotide sequence of SEQ ID NOs: 1 to 4.
12. The GPD1 inhibitor for use of any one of embodiments 1 to 11, wherein said GPD1 inhibitor is coupled to a cell-penetrating peptide.
13. The GPD1 inhibitor for use of any one of embodiments 1 to 5, wherein said GPD1 inhibitor is (−)-epicatechin, (−)-epicatechin-3-gallate, (−)-epigallocatechin, (−)-epigallocatechin-3-gallate, preferably is (−)-epigallocatechin-3-gallate.
14. A medicament for treating and/or preventing cancer comprising (i) a GPD1 inhibitor and (ii) a pharmaceutically acceptable carrier.
15. A kit comprising a GPD1 inhibitor comprised in a housing.
16. The kit of embodiment 15, further comprising a means of administration for said GPD1 inhibitor.
17. The kit of embodiment 15 or 16, further comprising a cancer therapeutic agent.
18. A method for identifying a subject suffering from cancer susceptible to cancer treatment by administration of a GPD1 inhibitor comprising
a) determining in a sample of said subject the amount of a GPD1 gene product,
b) comparing said amount determined in step a) to a reference, and
c) based on the result of step b), identifying a subject susceptible to cancer treatment by administration of a GPD1 inhibitor.
19. The method of embodiment 18, wherein said subject is a subject suffering from brain cancer, preferably a glioma, more preferably an astrocytoma, an oligodendroglioma or an oligoastocytoma, most preferably a glioblastoma multiforme.
20. The method of embodiment 18 or 19, wherein said subject is a subject being administered or having been administered a cancer therapeutic agent.
21. The method of embodiment 20, wherein said administering a cancer therapeutic agent is chemotherapy.
22. A method for treating and/or preventing cancer in a subject, comprising administering to said subject a GPD1 inhibitor.
23. The method of embodiment 22, wherein said subject is a subject known or suspected to suffer from cancer or is a subject at risk of developing cancer.
24. The method of embodiment 22 or 23, wherein said cancer is brain cancer or bladder cancer.
25. The method of any one of embodiments 22 to 24, wherein said brain cancer is a glioma, more preferably an astrocytoma, an oligodendroglioma or an oligoastocytoma, most preferably a glioblastoma multiforme.
26. The method of any one of embodiments 22 to 25, wherein said treatment of cancer further comprises administration of a cancer therapeutic agent, preferably comprises administration of chemotherapy.
27. The method of any one of embodiments 22 to 26, wherein administering a GPD1 inhibitor is administering an effective dose of a GPD1 inhibitor.
28. The method of any one of embodiments 22 to 27, wherein said GPD1 inhibitor is comprised in a pharmaceutical composition.
29. A method for identifying a compound for treating and/or preventing cancer, comprising
a) contacting a reaction mixture comprising a GPD1 polypeptide, glycerol-3-phosphate, and nicotine-adenine-dinucleotide (NAD) with a compound suspected to have the activity of being a direct GPD1 inhibitor,
b) further contacting said reaction mixture of a) with a luciferase and a luciferase substrate,
c) determining (i) NADH absorbance and/or (ii) the luciferase-mediated luminescence in the reaction mixture, and
d) identifying a compound for treating and/or preventing cancer based on the result of the determination step of c).
30. The method of embodiment 29, wherein said contacting in step b) is performed at a pH of 9±0.5, preferably wherein said contacting in steps a) and b) is performed at a pH of 9±0.5.
31. The method of embodiment 29, wherein a compound having the activity of being a GPD1 inhibitor is identified as a compound for treating and/or preventing cancer.

All references cited in this specification are herewith incorporated by reference with respect to their entire disclosure content and the disclosure content specifically mentioned in this specification.

### FIGURE LEGENDS

FIG. 1: GPD1 expression in brain tumor stem cells (BTSCs) and in highly tumorigenic human glioblastoma multiforme (GBM) cell lines; anti-GPD1 Western blot on SDS-PAGE separated cellular proteins of the indicated cells. NSC: neural stem cells; Turn: BTSCs; LN and U87MG: GBM cell lines.

FIG. 2: GPD1 KO tumor stem cells fail to form neurospheres. Phase contrast photographs of wildtype brain tumor stem cells (WT BTSCs, upper row) and of mouse brain tumor stem cells (mBTSCs) in which GPD1 expression was knocked out (lower row). Cells were seeded at four different densities and photographed at two different time points after seeding as indicated.

FIG. 3: Survival following GPD1-KD

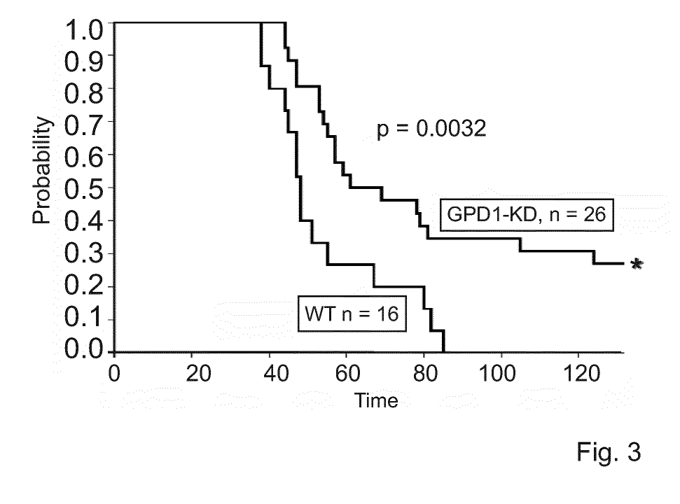

Survival study of tumor-bearing mice w/o GPD1-KD. The tumors were induced at birth. At the age of four weeks tamoxifen was injected for ten days to permanently activate the GPD1 miRNA and the symptom free time was measured. Mice with a GPD1-KD survived significantly longer than mice with a WT tumor

FIG. 4: Loss of GPD1 reduces the G3P levels to that of normal neural stem cells

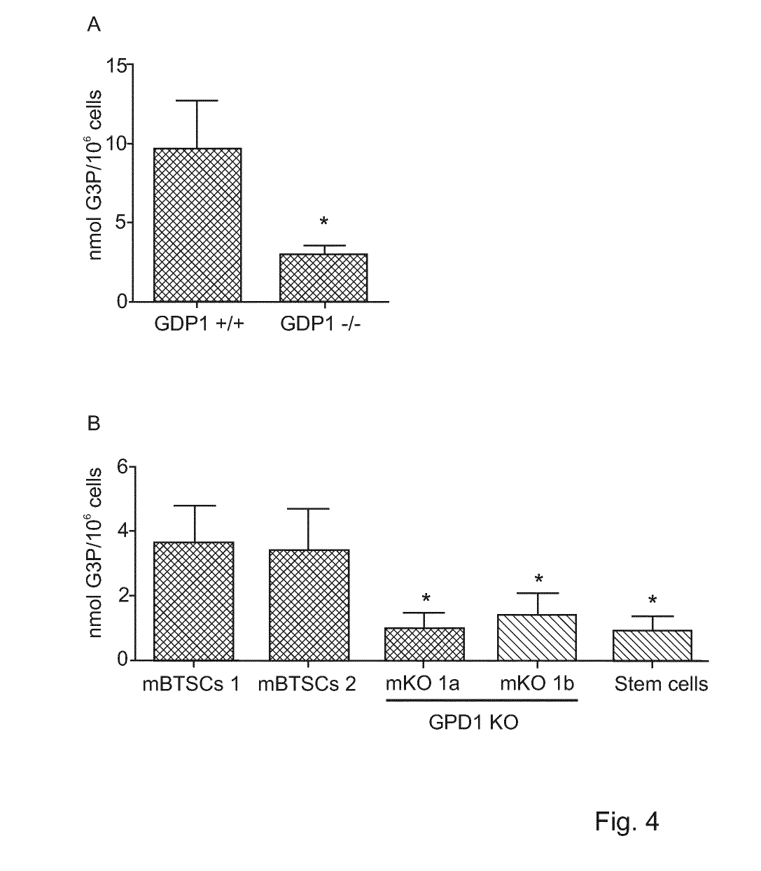

Intracellular levels of glycerol-3-phosphate (G3P) per 10⁶ cells in A) U87MG human glioblastoma cells; and in B) murine brain tumor stem cells (mBTSCs) and neural stem cell (Stem cells) lines; mKO1a and mKO1b indicate different guide RNAs used in BTSCs.

FIG. 5: Effects of GPD1 KO on lipid storage. Murine brain tumor stem cells (mBTSCs) and neural stem cell (Stem cells) lines were stained with Oil Red O; mKO#1 and mKO#2 indicate different guide RNAs used in BTSCs. Arrows point to exemplary lipid droplets.

FIG. 6: Sensitivity of GPD1 KO cells to Chemotherapy. % apoptotic cells after 72 h treatment with Temozolomide.

The following Examples shall merely illustrate the invention. They shall not be construed, whatsoever, to limit the scope of the invention.

### EXAMPLE 1: EXPRESSION OF GPD1 IN TUMOR CELLS AND IN TUMOR STEM CELLS

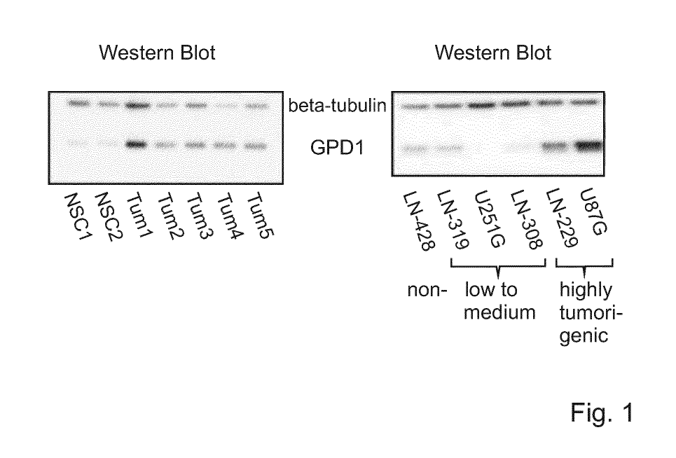

High GPD1 protein levels could be detected in mouse BTSCs; High GPD1 protein levels also correlated with high tumorigenicity of human glioma cell lines, in particular LN-229 and U87G (FIG. 1).

### EXAMPLE 2: GPD1 EXPRESSION CO-LOCALIZES WITH STEM CELL MARKER TLX

In primary mouse brain tumors, GPD1 positive cells were found to form clusters at the tumor edge and these clusters did not overlap with PCNA staining. GPD1 expression was found in T1x-GFP expressing BTSCs, which are PCNA negative.

### EXAMPLE 3: GPD1 KO PROMOTES DIFFERENTIATION

GPD1 was knocked out in primary mBTSCs and cells were allowed to differentiate in vitro. GPD1 KO BTSCs expressed significantly more neuronal cell markers (9%), compared to WT tumors (1%); also, WT BTSCs were more likely to differentiate into oligodendrocytes (consistent with GBM) compared to GPD1 KO cells (7% vs. <1%).

### EXAMPLE 4: GPD1 KO TUMOR STEM CELLS FAIL TO FORM NEUROSPHERES

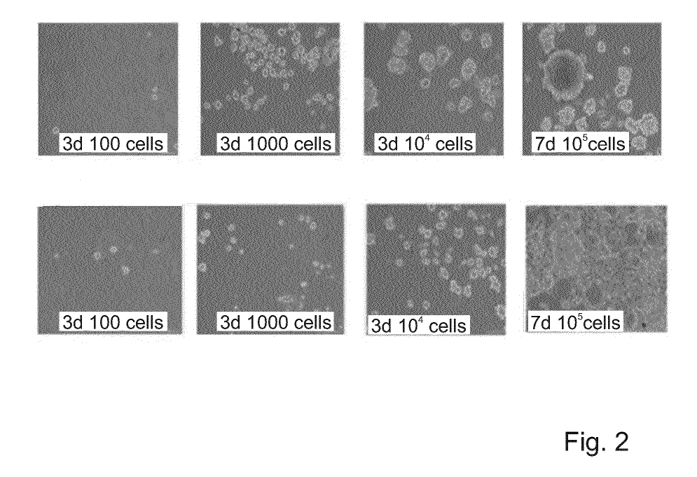

Stem cells isolated from tumors were cultured as neurospheres in vitro (FIG. 2). GPD1 KO tumor stem cells formed less and smaller neurospheres and instead grew as attached cells.

### EXAMPLE 5: INDUCIBLE KNOCKDOWN OF GPD1 IN PRIMARY BRAIN TUMORS PROLONGS SURVIVAL IN VIVO

Animals with existing primary brain tumors were treated with Tamoxifen (QDx10) to induce the expression of a miRNA against GPD1. Animals with GPD1 knockdown showed significant increase in survival.

### EXAMPLE 6: LOSS OF GPD1 REDUCES THE G3P LEVELS TO THAT OF NORMAL NEURAL STEM CELLS

Intracellular G3P was measured using a commercially available kit.

### EXAMPLE 7: EFFECTS OF GPD1 KO ON LIPID STORAGE (OILREDO STAINING)

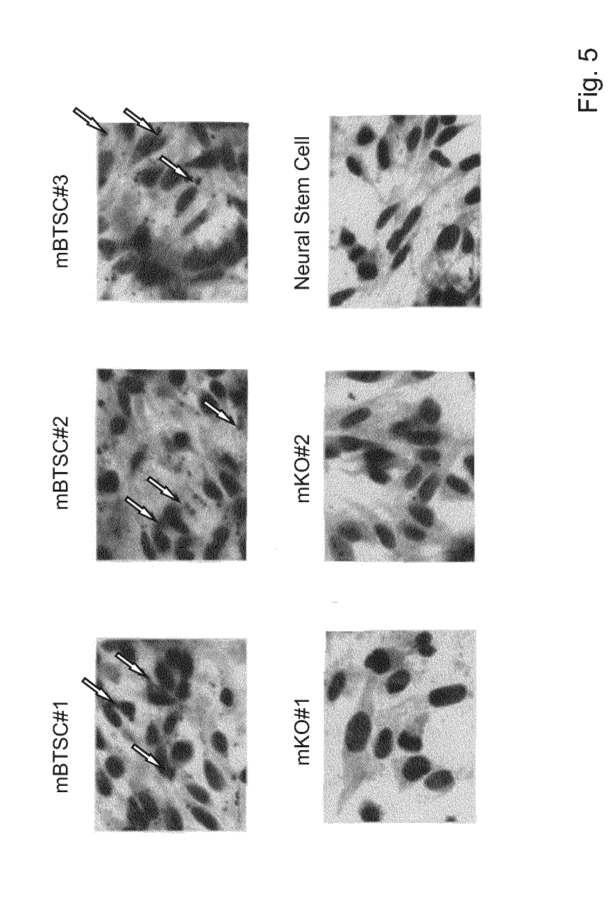

GPD1 KO phenotype shows lipid storage comparable to normal neural stem cells (FIG. 5). In contrast, mBTSCs showed increased lipid storage.

### EXAMPLE 8: SENSITIVITY OF GPD1 KO CELLS TO CHEMOTHERAPY

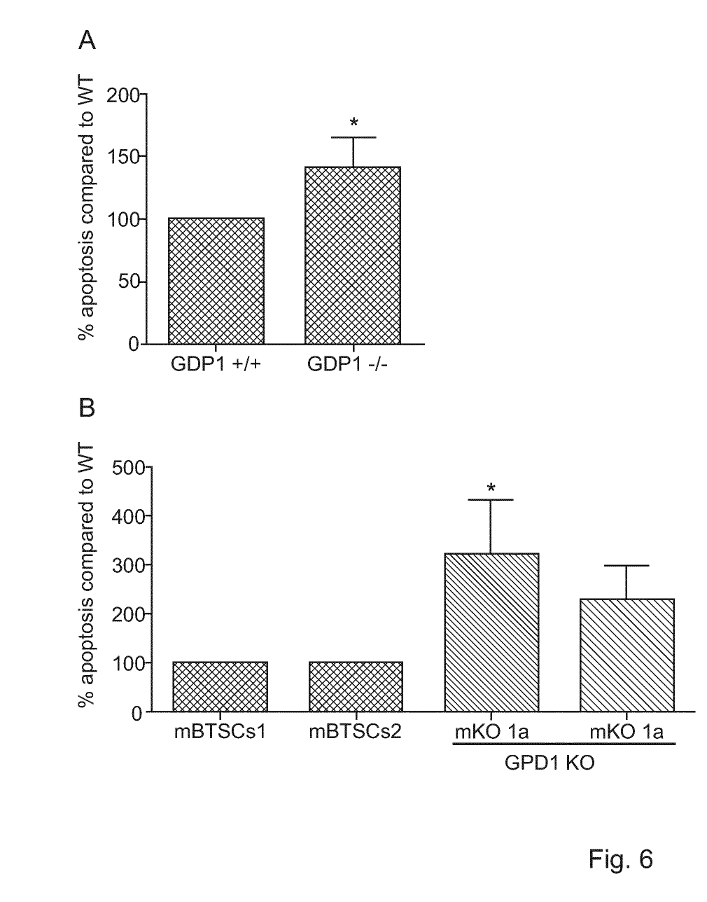

U87MG, mBTSCs or the corresponding GPD1 KO cells were treated with 250 μM Temozolomide for 72 hours. An apoptosis assay showed that GPD1 KO cells are more sensitive to chemotherapy than the BTSC or human U87MG cell line (FIG. 6).

1. A method for treating and/or preventing cancer in a subject, comprising administering to said subject an inhibitor of glycerol-3-phosphate dehydrogenase 1 (GPD1 inhibitor).

2. The method of claim 1, wherein said GPD1 inhibitor is a direct GPD1 inhibitor, preferably is a polypeptide or polynucleotide specifically binding and inhibiting GPD1.

3. The method of claim 1, wherein said GPD1 inhibitor is selected from the group consisting of an antibody, an aptamer, an anticalin, and a Designed Ankyrin Repeat Protein (DARPin).

4. The method of claim 1, wherein said GPD1 inhibitor is an indirect GPD1 inhibitor.

5. The method of claim 4, wherein said GPD1 inhibitor is selected from the group consisting of a shRNA, a siRNA, a miRNA agent, an antisense molecule, an antisense oligonucleotide, a ribozyme, and a pair of CRISPR/Cas oligonucleotides.

6. The method of claim 1, wherein said GPD1 inhibitor is (−)-epicatechin, (−)-epicatechin-3-gallate, (−)-epigallocatechin, (−)-epigallocatechin-3-gallate, preferably is (−)-epigallocatechin-3-gallate.

7. The method of claim 1, wherein said cancer is bladder cancer or brain cancer, wherein said brain cancer preferably is a glioma, more preferably an astrocytoma, an oligodendroglioma or an oligoastrocytoma, most preferably a glioblastoma multiforme.

8. The method of claim 1, wherein said treating and/or preventing cancer further comprises administration of a cancer therapeutic agent, preferably comprises administration of chemotherapy.

9. (canceled)

10. (canceled)

11. A method for identifying a subject suffering from cancer susceptible to cancer treatment by administration of a GPD1 inhibitor comprising
a) determining in a sample of said subject the amount of a GPD1 gene product,
b) comparing said amount determined in step a) to a reference, and
c) based on the result of step b), identifying a subject susceptible to cancer treatment by administration of a GPD1 inhibitor.

12. The method of claim 11, wherein said subject is a subject suffering from brain cancer, wherein said brain cancer preferably is a glioma, more preferably an astrocytoma, an oligodendroglioma or an oligoastrocytoma, most preferably a glioblastoma multiforme.

13. The method of 11, wherein said GPD1 inhibitor comprises at least one nucleic acid comprising at least one sequence selected from SEQ ID Nos: 1 to 4.

14. (canceled)

15. A method for identifying a compound for treating and/or preventing cancer, comprising
a) contacting a reaction mixture comprising a GPD1 polypeptide, glycerol-3-phosphate, and nicotine-adenine-dinucleotide (NAD) with a compound suspected to have the activity of being a direct GPD1 inhibitor,
b) further contacting said reaction mixture of a) with a luciferase and a luciferase substrate,
c) determining (i) NADH absorbance and/or (ii) the luciferase-mediated luminescence in the reaction mixture, and
(d) identifying a compound for treating and/or preventing cancer based on the result of the determination step of c).

16. The method of claim 4, wherein said indirect GPD1 inhibitor is a polypeptide comprising a lysosome-degradation sequence.

17. The method of claim 4, wherein said indirect GPD1 inhibitor is a chaperone-mediated autophagy-targeting motif (CTM) or is a polynucleotide.

18. The method of claim 4, wherein said GPD1 inhibitor comprises at least one nucleic acid comprising at least one sequence selected from SEQ ID NOs: 1 to 4.
